(19) United States
(12) Patent Application Publication
(10) Pub. No.: US 2016/0222024 A1
(43) Pub. Date: Aug. 4, 2016

(54) S-ENANTIOMER OF TETRACYCLIC INDOLE DERIVATIVE AS PBR LIGANDS

(71) Applicant: GE HEALTHCARE LIMITED, Little Chalfont, Buckinghamshire (GB)

(72) Inventors: William John Trigg, Little Chalfont, Buckinghamshire (GB); Paul Alexander Jones, Little Chalfont, Buckinghamshire (GB)

(21) Appl. No.: 15/021,877

(22) PCT Filed: Sep. 19, 2014
(86) PCT No.: PCT/EP2014/069976

§ 371 Date: Mar. 14, 2016

(30) Foreign Application Priority Data
Sep. 20, 2013 (GB) 1316764.8

(51) Int. Cl. C07D 495/04; B01J 19/24; A61K 51/04

(52) U.S. Cl. CPC C07D 495/04; A61K 51/0446; B01J 19/24; B01J 2219/24

(57) ABSTRACT

The present invention concerns in vivo imaging and in particular in vivo imaging of translocator protein (TSPO, formerly known as the peripheral benzodiazepine receptor). An indole-based in vivo imaging agent is provided that overcomes problems relating to known TSPO-binding radiotracers. The present invention also provides a precursor compound useful in the synthesis of the in vivo imaging agent of the invention, as well as a method for synthesis of said precursor compound. Other aspects of the invention include a method for the synthesis of the in vivo imaging agent of the invention comprising use of the precursor compound of the invention, a kit for carrying out said method, and a cassette for carrying out an automated version of said method. In addition, the invention provides a radiopharmaceutical composition comprising the in vivo imaging agent of the invention, as well as methods for the use of said in vivo imaging agent.

### TECHNICAL FIELD OF THE INVENTION

The present invention concerns in vivo imaging and in particular in vivo imaging of translocator protein (TSPO, formerly known as the peripheral benzodiazepine receptor). An indole-based in vivo imaging agent is provided that overcomes problems relating to known TSPO-binding radiotracers. The present invention also provides a precursor compound useful in the synthesis of the in vivo imaging agent of the invention, as well as a method for synthesis of said precursor compound. Other aspects of the invention include a method for the synthesis of the in vivo imaging agent of the invention comprising use of the precursor compound of the invention, a kit for carrying out said method, and a cassette for carrying out an automated version of said method. In addition, the invention provides a radiopharmaceutical composition comprising the in vivo imaging agent of the invention, as well as methods for the use of said in vivo imaging agent.

### DESCRIPTION OF RELATED ART

TSPO is known to be mainly localised in peripheral tissues and glial cells but its physiological function remains to be clearly elucidated. Subcellularly, TSPO is known to localise on the outer mitochondrial membrane, indicating a potential role in the modulation of mitochondrial function and in the immune system. It has furthermore been postulated that TSPO is involved in cell proliferation, steroidogenesis, calcium flow and cellular respiration.

In studies examining the expression of TSPO in normal and diseased tissue, Cosenza-Nashat et al (2009 Neuropathol Appl Neurobiol; 35(3): 306-328) confirmed that TSPO expression in normal brain is minimal. This same paper demonstrated that in disease states elevated TSPO was present in parenchymal microglia, macrophages and some hypertrophic astrocytes, but the distribution of TSPO varied depending on the disease, disease stage and proximity to the lesion or relation to infection. Microglia and macrophages are the predominant cell type expressing TSPO in diseased brains and astrocytes can also express TSPO in humans.

Positron emission tomography (PET) imaging using the TSPO selective ligand, (R)-[¹¹C]PK11195 has been widely used as a generic indicator of central nervous system (CNS) inflammation. However, there are limitations with (R)-[¹¹C]PK11195 including high nonspecific binding, low brain penetration, high plasma protein binding, and a difficult synthesis. Furthermore, the role of its radiolabelled metabolites is not known, and quantification of binding requires complex modeling.

Prompted by the issues with (R)-[¹¹C]PK11195, a next generation of TSPO-binding PET tracers has been developed leading to some demonstrating higher specific to non-specific signals and higher brain uptake, including [¹⁸F]-FEPPA, [¹⁸F] PBR111, [¹¹C]-PBR28, [¹¹C]-DPA713, [¹¹C]-DAA1106, and [¹¹C]-AC-5126 (Chauveau et al 2008 Eur J Nucl Med Mol Imaging; 35: 2304-2319). However, more recently, intra-subject variability in PET results has been observed in this new generation of tracers. These tracers bind TSPO in brain tissue from different subjects in one of three ways. High-affinity binders (HABs) and low-affinity binders (LABs) express a single binding site for TSPO with either high or low affinity, respectively. Mixed affinity binders (MABs) express roughly equal numbers of the HAB and LAB binding sites (Owen et al 2011 J Nucl Med; 52: 24-32). Owen et al (J Cerebral Blood Flow Metab 2012; 32: 1-5) demonstrated that a polymorphism in TSPO (Ala147Thr) is responsible for the observed intra-subject variability in binding.

Fujita et al (Neuroimage 2008; 40: 43-52) carried out [¹¹C]PBR28 imaging in healthy volunteers and noted that 2 out of the 12 subjects imaged had a time course of brain activity that could have been mimicked by the absence or blockade of TSPO. Whole body imaging of these 2 subjects showed negligible binding to kidneys, lungs and spleen so that they appeared to lack the binding site of [¹¹C]PBR28 or lack TSPO receptors.

In another study examining in vivo imaging of [¹¹C]PBR28 (Kreisl et al NeuroImage 2010; 49: 2924-2932), uptake in organs with high densities of TSPO was shown to be 50% to 75% lower in LABs than in HABs, whereas for [¹¹C]PK11195 differences in uptake were only seen in heart and lung. [³H]PBR28 in an in vitro assay showed more than 10-fold lower TSPO affinity in LABs than in HABs. In monkeys, in vivo specific binding of [¹¹C]PK11195 in monkey brain was ˜80-fold lower than that reported for [¹¹C]PBR28. These results supported a conclusion that non-binding of [¹¹C]PBR28 in LABs was due to low affinity for TSPO, and that the relatively low in vivo specific binding of [¹¹C]PK11195 may have obscured its detection of nonbinding in peripheral organs.

Mizrahi et al (2012 J Cerebral Blood Flow Metabol; 32: 968-972) demonstrated that [¹⁸F]FEPPA demonstrates clear differences in the in vivo imaging characteristics between binding groups.

The presence HABs, MABs and LABs presents a problem for the utility of TSPO radioligands because the signal cannot reliably be interpreted. It would be desirable to develop a strategy that overcomes this problem.

### SUMMARY OF THE INVENTION

The present invention provides a compound that binds to TSPO and has improved properties compared with known TSPO-binding compounds. In particular, the compound of the present invention addresses the issue of heterogenous binding in HABs, MABs and LABs.

### DESCRIPTION OF THE PREFERRED EMBODIMENTS

In one aspect, the present invention provides a compound of the following structure:

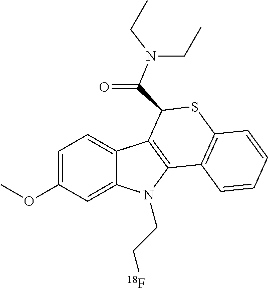 [SMILES: CCN(CC)C(=O)[C@H]1Sc2ccccc2-c2c1c1ccc(OC)cc1n2CC[18F]]

or a salt or solvate thereof.

Suitable salts according to the invention, include physiologically acceptable acid addition salts such as those derived from mineral acids, for example hydrochloric, hydrobromic, phosphoric, metaphosphoric, nitric and sulphuric acids, and those derived from organic acids, for example tartaric, trifluoroacetic, citric, malic, lactic, fumaric, benzoic, glycolic, gluconic, succinic, methanesulphonic, and para-toluenesulphonic acids.

Suitable solvates according to the invention include ethanol, water, saline, physiological buffer and glycol.

The synthesis of the compound of the invention may be based on the methods described by Okubo et al (Bioorg Med Chem 2004; 12: 3569-80). Example 2 below describes how a non-radioactive version of Compound 1 of the invention was obtained. The enantiomers were resolved using the method described in Example 13 of WO 2010/109007.

In another aspect the present invention provides a precursor compound for use in the preparation of the compound of the invention wherein said precursor compound is of Formula I:

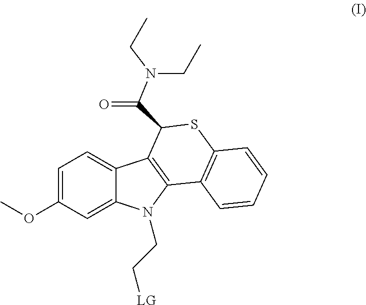

or a salt or solvate thereof;
wherein LG is a leaving group.

A “leaving group” in the context of the present invention refers to an atom or group of atoms that is displaced as a stable species during a substitution or displacement radiofluorination reaction. Examples of suitable leaving groups are the halogens chloro, bromo and iodo, and the sulfonate esters mesylate, tosylate nosylate and triflate. In one embodiment, said leaving group is selected from mesylate, tosylate and triflate, and is preferably mesylate.

In another aspect the present invention provides a method to prepare the compound of the invention wherein said method comprises reacting the precursor compound of Formula I as defined herein with a suitable source of [¹⁸F]fluoride to obtain said compound.

The term “suitable source of [¹⁸F]fluoride” means [¹⁸F]fluoride in a chemical form that replaces LG in a nucleophilic substitution reaction. [¹⁸F]-fluoride ion (¹⁸F) is normally obtained as an aqueous solution from the nuclear reaction ¹⁸O(p,n)¹⁸F and typically made reactive by the addition of a cationic counterion and the subsequent removal of water.

Suitable cationic counterions should possess sufficient solubility within the anhydrous reaction solvent to maintain the solubility of [¹⁸F]fluoride. Counterions that are typically used include large but soft metal ions such as rubidium or caesium, potassium complexed with a cryptand such as Kryptofix™ 2.2.2 (K222), or tetraalkylammonium salts. A preferred counterion is potassium complexed with a cryptand such as K222 because of its good solubility in anhydrous solvents and enhanced [¹⁸F]fluoride reactivity.

A more detailed discussion of well-known ¹⁸F labelling techniques can be found in Chapter 6 of the “Handbook of Radiopharmaceuticals” (2003; John Wiley and Sons: M. J. Welch and C. S. Redvanly, Eds.).

In a preferred embodiment, the method to prepare a compound of Formula I of the invention is automated. [¹⁸F]-radiotracers may be conveniently prepared in an automated fashion by means of an automated radiosynthesis apparatus. There are several commercially-available examples of such apparatus, including Tracerlab MX™ and FASTlab™ (GE Healthcare), FDGPlus Synthesizer (Bioscan) and Synthera® (IBA). Such apparatus commonly comprises a “cassette” (sometimes referred to as a “cartridge”), often disposable, in which the radiochemistry is performed, which is fitted to the apparatus in order to perform a radiosynthesis. The cassette normally includes fluid pathways, a reaction vessel, and ports for receiving reagent vials as well as any solid-phase extraction cartridges used in post-radiosynthetic clean up steps.

The present invention provides in another aspect a cassette for carrying out the automated method of the invention wherein said cassette comprises:
(i) a vessel containing the precursor compound as defined herein; and,
(ii) means for eluting the vessel of step (i) with a suitable source of [¹⁸F]fluoride.

The cassette of the invention may optionally additionally comprise:
(iii) an ion-exchange cartridge for removal of excess [¹⁸F]fluoride; and/or
(iv) one or more solid phase extraction cartridges for purification of the [¹⁸F] labelled reaction mixture.

For the cassette of the invention, the suitable and preferred embodiments of the precursor compound of Formula I and suitable source of [¹⁸F]fluoride are as previously defined herein.

Another aspect of the invention is a radiopharmaceutical composition comprising the compound of the invention together with a biocompatible carrier in a form suitable for mammalian administration. The “biocompatible carrier” is a fluid, especially a liquid, in which the compound of the invention is suspended or dissolved, such that the composition is physiologically tolerable, i.e. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g. salts of plasma cations with biocompatible counterions), sugars (e.g. glucose or sucrose), sugar alcohols (e.g. sorbitol or mannitol), glycols (e.g. glycerol), or other non-ionic polyol materials (e.g. polyethyleneglycols, propylene glycols and the like). The biocompatible carrier may also comprise biocompatible organic solvents such as ethanol. Such organic solvents are useful to solubilise more lipophilic compounds or formulations. Preferably the biocompatible carrier is pyrogen-free water for injection, isotonic saline or an aqueous ethanol solution. The pH of the biocompatible carrier for intravenous injection is suitably in the range 4.0 to 10.5.

The pharmaceutical composition may optionally contain further ingredients such as buffers; pharmaceutically acceptable solubilisers (e.g. cyclodextrins or surfactants such as Pluronic, Tween or phospholipids); pharmaceutically acceptable stabilisers or antioxidants (such as ethanol, ascorbic acid, gentisic acid or para-aminobenzoic acid).

The radiopharmaceutical composition may be administered parenterally, i.e. by injection. Where the compound of the invention is provided as a radiopharmaceutical composition, the method for preparation of said compound suitably further comprises steps including removal of organic solvent, addition of a biocompatible buffer and any optional further ingredients. For parenteral administration, steps to ensure that the radiopharmaceutical composition is sterile and apyrogenic also need to be taken.

For the radiopharmaceutical composition of the invention, the suitable and preferred embodiments of the compound of the invention as defined herein.

The compound of the present invention has good binding affinity for TSPO. Therefore in a further aspect, the present invention provides an in vivo imaging method for determining the distribution and/or the extent of TSPO expression in a subject wherein said method comprises:
(i) administering to said subject the compound of the invention;
(ii) allowing said compound to bind to TSPO expressed in said subject;
(iii) detecting signals emitted by the radioisotope of said compound using positron-emission tomography (PET);
(iv) generating an image representative of the location and/or amount of said signals; and,
(v) determining the distribution and extent of TSPO expression in said subject wherein said expression is directly correlated with said signals emitted by said compound.

“Administering” the compound of the invention is preferably carried out parenterally, and most preferably intravenously. The intravenous route represents the most efficient way to deliver the in vivo imaging agent throughout the body of the subject and therefore into contact with TSPO expressed in said subject. Furthermore, intravenous administration does not represent a substantial physical intervention or a substantial health risk. The compound of the invention is preferably administered as the pharmaceutical composition of the invention, as defined herein. The in vivo imaging method of the invention can also be understood as comprising the above-defined steps (ii)-(v) carried out on a subject to whom the in vivo imaging agent of the invention has been pre-administered.

Following the administering step and preceding the detecting step, the compound of the invention is allowed to bind to TSPO. For example, when the subject is an intact mammal, the compound of the invention will dynamically move through the mammal's body, coming into contact with various tissues therein. Once the compound of the invention comes into contact with TSPO, a specific interaction takes place such that clearance of the compound of the invention from tissue with TSPO takes longer than from tissue without, or with less TSPO. A certain point in time will be reached when detection of compound specifically bound to TSPO is enabled as a result of the ratio between compound bound to tissue with TSPO versus that bound in tissue without, or with less TSPO. An ideal such ratio is around 2:1.

The “detecting” step of the method of the invention involves detection of signals emitted by the radioisotope by means of a detector sensitive to said signals. This detection step can also be understood as the acquisition of signal data. Positron-emission tomography (PET) is a suitable in vivo imaging procedure for use in the method of the invention.

The “generating” step of the method of the invention is carried out by a computer which applies a reconstruction algorithm to the acquired signal data to yield a dataset. This dataset is then manipulated to generate images showing the location and/or amount of signals emitted by said radioisotope. The signals emitted directly correlate with the expression of TSPO such that the “determining” step can be made by evaluating the generated image.

The “subject” of the invention can be any human or animal subject. Preferably the subject of the invention is a mammal. Most preferably, said subject is an intact mammalian body in vivo. In an especially preferred embodiment, the subject of the invention is a human. The in vivo imaging method may be used to study TSPO in healthy subjects, or in subjects known or suspected to have a pathological condition associated with abnormal expression of TSPO (hereunder a “TSPO condition”). Preferably, said method relates to the in vivo imaging of a subject known or suspected to have a TSPO condition, and therefore has utility in a method for the diagnosis of said condition.

Examples of such TSPO conditions where in vivo imaging would be of use include multiple sclerosis, Rasmeussen's encephalitis, cerebral vasculitis, herpes encephalitis, AIDS-associated dementia, Parkinson's disease, corticobasal degeneration, progressive supranuclear palsy, multiple system atrophy, Huntington's Disease, amyotrophic lateral sclerosis, Alzheimer's disease, ischemic stroke, peripheral nerve injury, epilepsy, traumatic brain injury, acute stress, chronic stress, neuropathic pain, lung inflammation, chronic obstructive pulmonary disease, asthma, inflammatory bowel disease, rheumatoid arthritis, primary fibromyalgia, nerve injury, atherosclerosis, kidney inflammation, ischemia-reperfusion injury, and cancer, in particular cancer of the colon, prostate or breast.

In an alternative embodiment, the in vivo imaging method of the invention may be carried out repeatedly during the course of a treatment regimen for said subject, said regimen comprising administration of a drug to combat a TSPO condition. For example, the in vivo imaging method of the invention can be carried out before, during and after treatment with a drug to combat a TSPO condition. In this way, the effect of said treatment can be monitored over time. PET has excellent sensitivity and resolution, so that even relatively small changes in a lesion can be observed over time, which is particularly advantageous for treatment monitoring.

In an alternative aspect, the present invention provides said compound of the invention for use in an in vivo imaging method as defined herein.

In another alternative aspect, the present invention provides the compound of the invention as defined herein for use in the manufacture of a radiopharmaceutical composition as defined herein for use in an in vivo imaging method as defined herein.

In a yet further aspect, the present invention provides a method for diagnosis of a condition in which TSPO is upregulated, said method comprising the in vivo imaging method as defined herein, together with a further step (vi) of attributing the distribution and extent of TSPO expression to a particular clinical picture.

In an alternative aspect, the present invention provides the compound of the invention as defined herein for use in the method for diagnosis as defined herein.

In another alternative aspect, the present invention provides the compound of the invention as defined herein for use in the manufacture of a radiopharmaceutical composition as defined herein for use in the method for diagnosis as defined herein.

The invention is now illustrated by a series of non-limiting examples.

### BRIEF DESCRIPTION OF THE EXAMPLES

Example 1 describes the prior art compounds used to compare with compounds of the present invention.

Example 2 describes the synthesis of non-radioactive Compound 1 of the invention.

Example 3 describes the testing of racemates in the binder/non-binder assay.

Example 4 describes the testing of resolved enantiomers in the binder/non-binder assay.

### LIST OF ABBREVIATIONS USED IN THE EXAMPLES

DCM dichloromethane

DMF dimethylformamide

h hour(s)

IPA isopropyl alcohol

LC-MS liquid chromatography mass spectrometry

MeOH methanol

NMR nuclear magnetic resonance

PEI polyetherimide

RT room temperature

SFC supercritical fluid chromatography

### EXAMPLES

### Example 1

### Prior Art Compounds

### Example 1(i)

### PK11195

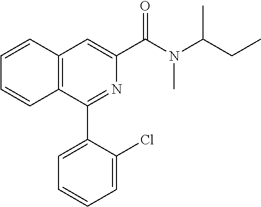 [SMILES: CCC(C)N(C)C(=O)c1cc2ccccc2c(-c2ccccc2Cl)n1]

PK11195 is commercially available.

### Example 1(ii)

### N-(2-methoxybenzyl)-N-(4-phenoxypyridin-3-yl)acetamide (PBR28)

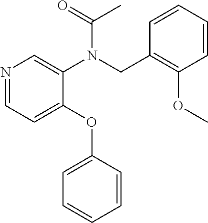 [SMILES: COc1ccccc1CN(C(C)=O)c1cnccc1Oc1ccccc1]

Non-radioactive PBR28 is commercially available.

### Example 1(iii)

### Non-radioactive 9-(2-Fluoro-ethyl)-5-methoxy-2,3,4,9-tetrahydro-1H-carbazole-4-carboxylic acid diethylamide (GE180)

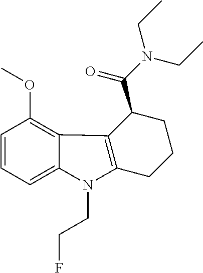 [SMILES: CCN(CC)C(=O)[C@H]1CCCc2c1c1c(OC)cccc1n2CCF]

A non-radioactive version of the prior art compound 9-(2-Fluoro-ethyl)-5-methoxy-2,3,4,9-tetrahydro-1H-carbazole-4-carboxylic acid diethylamide (known as GE180) was prepared for testing according to the method described by Wadsworth et al (2012 Bioorg Med Chem Letts; 22: 1308-1313) and in Examples 2 and 14 of WO 2010/109007.

### Example 2

### Synthesis of Non-Radioactive Compound 1

### Example 2(i)

### 4-oxothiochroman-2-carboxylic acid

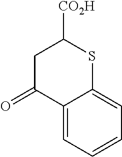 [SMILES: O=C1CC(C(=O)O)Sc2ccccc21]

A mixture of benzenethiol (82.6 g, 750 mmol, 77 mL) and furan-2,5-dione (73.5 g, 0.75 mol) in toluene (10 mL) was stirred at 50° C. for 40 min. After all materials were dissolved, triethylamine (363 mg, 3.6 mmol, 500 μL) in toluene (10 mL) was added over 10 min keeping the temperature below 70° C. After stirring at 70° C. for 20 min, the reaction mixture was concentrated in vacuo. The residue was dissolved in dichloromethane (150 mL) and the mixture cooled with an ice-cooling bath. Aluminum trichloride (150 g, 1.12 mmol) was added portion-wise keeping the temperature below 10° C. The reaction mixture was warmed up to RT and stirred for 1.5 h. A vigorous evolution of hydrogen chloride gas was observed. The reaction mixture was diluted in dichloromethane (150 mL) and slowly poured into vigorously stirred ice-cooling concentrated hydrochloric acid (500 mL). The dichloromethane layer was separated, dried over MgSO₄ and concentrated in vacuo to give a brown solid. The solid was triturated with diethyl ether and a yellow solid was collected by filtration to give 67.7 g (43%) of 4-oxothiochroman-2-carboxylic acid. The structure was confirmed by ¹H NMR (300 MHz; DMSO-d₆): δ_{H} 2.95-3.22 (2H, m, CH₂CHCO₂H), 4.40 (1H, dd, J=6 and 5 Hz, CH₂CHCO₂H), 7.18-7.57 (3H, m, CHCHCHCHC(S)) and 7.94 (1H, dd, J=8 and 1.5 Hz, CHCHCHCHC(S)).

### Example 2(ii)

### 4-oxothiochroman-2-carbonyl chloride

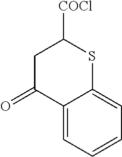 [SMILES: O=C1CC(C(=O)Cl)Sc2ccccc21]

4-oxothiochroman-2-carboxylic acid (15 g, 72.0 mmol) in dry dichloromethane (210 mL) was stirred under an atmosphere of nitrogen at RT for 18 h, with oxalyl chloride (18.2 g, 144.0 mmol, 12.6 mL) and one drop of dimethylformamide to catalyse the reaction. There was a vigorous evolution of gas as the solid dissolved. The reaction was then evaporated in vacuo to give 16.3 g (quantitative) of 4-oxothiochroman-2-carbonyl chloride as a gum that was used in the next step without purification. The structure was confirmed by ¹H NMR (300 MHz; CDCl₃): δ_{H} 3.15 (1H, dd, J=15 and 3 Hz, CH₂CHCO₂Cl), 3.35 (1H, dd, J=15 and 3 Hz, CH₂CHCO₂Cl), 4.33 (1H, t, J=6 Hz, CH₂CHCO₂Cl), 7.18-7.57 (3H, m, COCCHCHCHCH) and 7.94 (1H, dd, J=8 and 1.5 Hz, COCCHCHCHCH). ¹³C NMR (75 MHz; CDCl₃): δ_{C} 40.6, 53.0, 55.7, 111.3, 113.5, 131.4, 160.5, 161.8, 171.0 and 189.2.

### Example 2(iii)

### N,N-diethyl-4-oxothiochroman-2-carboxamide

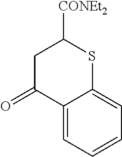

4-oxothiochroman-2-carbonyl chloride (16.3 g, 72.0 mmol), was dissolved in dichloromethane (210 mL) cooled to 0° C. Diethylamine (10.8 g, 147.4 mmol, 15 mL) in dichloromethane (40 mL) was then added dropwise over a period of 1 h. The reaction was allowed to warm to RT over a period of 1 h. The reaction mixture was quenched with a 5% potassium carbonate solution (100 mL) and extracted with dichloromethane. The combined organic layers were dried over MgSO₄ and concentrated in vacuo to give a dark green gum. The gum was then triturated with ethyl acetate and a solid was collected. 16 g (84%) of N,N-diethyl-4-oxothiochroman-2-carboxamide was obtained as brown crystals after purification by hot recrystallisation from ethyl acetate and petrol ether. The structure was confirmed by ¹H NMR (300 MHz; CDCl₃): δ_{H} 1.07 (3H, t, J=6 Hz, N(CH₂CH₃)ₐ), 1.24 (3H, t, J=6 Hz, N(CH₂CH₃)_{b}), 3.02-3.54 (6H, m, CH₂CHCO and N(CH₂CH₃)₂), 4.24-4.28 (1H, m, CH₂CHCO), 7.18-7.57 (3H, m, COCCHCHCHCH) and 7.94 (1H, dd, J=8 and 1.5 Hz, COCCHCHCHCH); ¹³C NMR (75 MHz; CDCl₃): δ_{C} 12.7, 14.6, 39.9, 40.6, 42.1, 125.6, 127.1, 128.6, 130.7, 137.8, 167.7 and 192.9.

LC-MS: m/z calcd for C₁₄H₁₇NO₂S 263.1; found, 264.0 (M+H)+.

### Example 2(iv)

### N,N-diethyl-9-methoxy-6,11-dihydrothiochromeno[4,3-b]indole-6-carboxamide and N,N-diethyl-7-methoxy-6,11-dihydrothiochromeno[4,3-b]indole-6-carboxamide

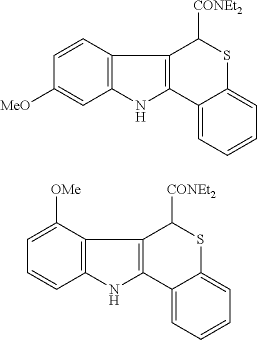

N,N-diethyl-4-oxothiochroman-2-carboxamide (3.3 g, 12.6 mmol) and 3-methoxyphenylhydrazine hydrochloride (3.3 g, 12.6 mmol in ethanol (10.5 mL) and concentrated sulfuric acid (1.9 mL, 34.7 mmol) were refluxed overnight. After cooling, the reaction mixture was filtered; the solid washed with ethanol to give 3.2 g (69%) of a mixture of N,N-diethyl-9-methoxy-6,11-dihydrothiochromeno[4,3-b]indole-6-carboxamide and N,N-diethyl-7-methoxy-6,11-dihydrothiochromeno[4,3-b]indole-6-carboxamide as a pale white solid. The structure was confirmed by ¹H NMR (300 MHz; DMSO-d₆): δ_{H} 0.90-1.00 (3H, m, N(CH₂CH₃)ₐ), 1.20-1.35 (3H, m, N(CH₂CH₃)_{b}), 3.10-3.30 (2H, m, N(CH₂CH₃)ₐ), 3.50-3.60 (2H, m, N(CH₂CH₃)_{b}), 3.80 (3H, s, OCH₃), 5.56 and 5.58 (1H, 2×s, CHCONEt₂), 6.45-7.30 (6H, m, ArH), 7.68-7.76 (1H, m, ArH), 11.50 (1H, br s, NH) and 11.62 (1H, br s, NH).####

LC-MS: m/z calcd for C₂₁H₂₂N₂O₂S 366.1; found, 367.0 (M+H)⁺.

### Example 2(v)

### N,N-diethyl-11-(2-fluoroethyl)-9-methoxy-6,11-dihydrothiochromeno[4,3-b]indole-6-carboxamide and N,N-diethyl-11-(2-fluoroethyl)-7-methoxy-6,11-dihydrothiochromeno[4,3-b]indole-6-carboxamide

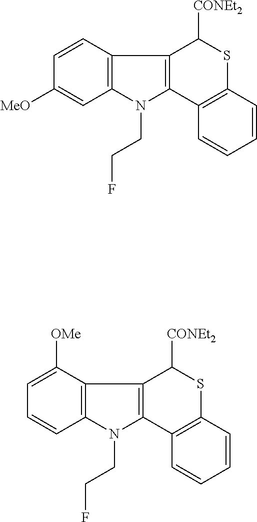

To a solution of mixture isomers, N,N-diethyl-9-methoxy-6,11-dihydrothiochromeno[4,3-b]indole-6-carboxamide and N,N-diethyl-7-methoxy-6,11-dihydrothiochromeno[4,3-b]indole-6-carboxamide (1.0 g, 2.7 mmol) in anhydrous DMF (10 mL), was added 2-fluoroethyl tosylate (1.2 g, 5.5 mmol) followed by sodium hydride (131 mg of a 60% dipersion in mineral oil, 5.5 mmol) under nitrogen. The reaction mixture was heated at 80° C. for 1 h. After cooling, the solvents were removed in vacuo, the residue quenched with water (30 mL), extracted with DCM (2×30 mL), dried (MgSO₄) and solvents removed in vacuo.

The residue was purified by silica gel chromatography eluting with DCM (A) and ethyl acetate (B) (5-10% B, 80 g, 5.0 CV, 60 mL/min) to afford 1.0 g (89%) of the isomer mixture as white foam. The mixture (400 mg) was then re-purified by semi preparative HPLC eluting with water (A) and methanol (B) (Gemini 5μ, C18, 110 A, 150×21 mm, 70-95% B over 20 min, 21 mL/min) to afford 240 mg (59%) of N,N-diethyl-11-(2-fluoroethyl)-9-methoxy-6,11-dihydrothiochromeno[4,3-b]indole-6-carboxamide as a yellow solid. The structure was confirmed by ¹H NMR (300 MHz, CDCl₃): δ_{H} 1.12 (3H, t, J=7 Hz, N(CH₂CH₃)ₐ), 1.35 (3H, t, J=7 Hz, N(CH₂CH₃)_{b}), 3.29-3.65 (4H, m, N(CH₂CH₃)₂), 3.88 (3H, s, OCH₃), 4.46-5.03 (4H, m, NCH₂CH₂F), 5.09 (1H, s, CHCONEt₂), 6.82 (1H, dd, J=9 and 2 Hz, 8-CH), 6.87 (1H, d, J=2 Hz, 10-CH), 7.14 (1H, dt, J=8 and 1 Hz, ArH), 7.26 (1H, dt, J=8 and 1 Hz, ArH), 7.31 (1H, d, J=9 Hz, 7-CH), 7.46 (1H, dd, J=8 and 1 Hz, ArH) and 7.55 (1H, d, J=8 Hz, ArH); ¹⁹F NMR (283 MHz, CDCl₃): δ_{F}−219.5.

LC-MS: m/z calcd for C₂₃H₂₅FN₂O₂S 412.2; found, 413.1 (M+H)⁺.

Further elution afforded 100 mg (25%) of N,N-diethyl-11-(2-fluoroethyl)-7-methoxy-6,11-dihydrothiochromeno[4,3-b]indole-6-carboxamide as a white solid. The structure was confirmed by ¹H NMR (300 MHz, CDCl₃): δ_{H} 1.04 (3H, t, J=7 Hz, N(CH₂CH₃)ₐ), 1.40 (3H, t, J=7 Hz, N(CH₂CH₃)_{b}), 3.23-3.71 (4H, m, N(CH₂CH₃)₂), 3.88 (3H, s, OCH₃), 4.45-5.00 (4H, m, NCH₂CH₂F), 5.53 (1H, s, CHCONEt₂), 6.52 (1H, d, J=8 Hz, 8-CH), 7.00 (1H, d, J=8 Hz, 10-CH), 7.10-7.17 (2H, m, 9-CH and ArH), 7.25 (1H, dt, J=8 and 1 Hz, ArH), 7.42 (1H, dd, J=8 and 1 Hz, ArH) and 7.59 (1H, d, J=8 Hz, ArH); ¹⁹F NMR (283 MHz, CDCl₃): δ_{F}−220.0.

LC-MS: m/z calcd for C₂₃H₂₅FN₂O₂S 412.2; found, 413.1 (M+H)⁺.

SFC chiral separation was used to separate out the S-enantiomer using the following conditions:

CO₂: AGA SFC grade
Analytical column: Whelk-01 10 × 250 mm, 5 μm, 100 Å
Flow: 13 ml/min
Pressure: 100 bar
Temp: 40° C.
Eluent: 40% Methanol
Injection concentration: 102 mg/ml
Injection solvent: MeOH:IPA 1:1
Injection volume: 100 μL

S-enatiomer: Retention time: 7.3 min, purity 95%

R-enatiomer: Retention time: 9.1 mM, purity 99%

### Example 3

### Binder/Non-Binder Assay of Racemates

Membrane protein was prepared from human platelets obtained from 4 donor whole blood samples. Two of these donor samples were previously identified as having high affinity and 2 identified as having low affinity based on PBR28 binding affinity. Platelet pellets were homogenized in 10 ml buffer 1 (0.32 mM sucrose, 5 mM Tris base, 1 mM MgCl₂, pH 7.4, 4° C.). The homogenates were centrifuged at 48,000×g for 15 minutes at 4° C. in a Beckman J2-MC centrifuge. The supernatant was removed and pellets were re-suspended in at least 10 ml buffer 2 (50 mM Tris base, 1 mM MgCl₂, pH 7.4, 4° C.) and washed by centrifugation at 48,000×g for 15 mM at 4° C. in buffer 2. Membranes were suspended in 2 ml buffer 2 and the protein concentration was determined using Protein Assay Kit II (Bio Rad cat #500-0002). Aliquots were stored at −80° C. until use.

Aliquots of membrane suspension were thawed and homogenized with assay buffer 3 (50 mM Tris base, 140 mM NaCl, 1.5 mM MgCl₂, 5 mM KCl, 1.5 mM CaCl₂, pH 7.4, 37° C.). For competitive binding experiments, non-labelled PBR28 (ABX cat #1653) or PK11195 was diluted on a Beckman Biomek 2000 workstation at 11 serial dilutions ranging from 100 μM to 1 nM and added to a non-binding 96 well microplate containing 5 nM [³H]PK11195 (Perkin Elmer Cat #NET885001MC). Compound 1 was diluted on a Beckman Biomek 2000 workstation at 11 serial dilutions ranging from 1 μM to 0.01 nM. GE180 was diluted at 11 serial dilutions ranging from 100 μM to 1 nM. Total and nonspecific binding assessments were also performed. 160 μL of platelet membranes diluted to 30 μg/mL were added to the assay plate for a final volume of 200 μL/well. Assay plates were incubated at 37° C. for at least one hour with termination of incubation by filtering onto GF/B glass fiber plates (Perkin Elmer; cat #6005177) pre-soaked in 0.1% PEI in saline for 60 minutes. Assay plates were rinsed five to six times with ice cold buffer 4 (50 mM Tris Base, 1.4 mM MgCl₂, pH 7.4, 4° C.) on a Perkin Elmer Filtermate 196. Plates were then dried, the bottoms sealed, and 50 μL of MicroScint 20 (Perkin Elmer cat #6013621) was added to each well. After sealing the tops, the plates were allowed to equilibrate for at least 30 minutes and the captured radioactivity was counted on a Perkin Elmer TopCount NTX. Compound 1 was used as racemate. The compounds were tested in triplicate in the [3H]PK11195 competitive binding assay and the affinity of the compounds was determined by analyzing the data using GraphPad Prism 5.0 and the low:high affinity ratios were calculated.

| | Low Affinity Site | High Affinity Site | |
|---|---|---|---|
| Compound | (nM) | (nM) | Low:High |
| GE180 | 37.87 | 2.45 | 15.44 |
| Compound 1 | 0.51 | 0.05 | 9.87 |

### Example 4

### Binder/Non-Binder Assay for Resolved Enantiomers

Compound 1 was resolved into enantiomers as described in Example 2 and the competitive binding assay was performed using platelets isolated from the same 4 human donor whole blood samples. The same assay procedure as in Example 3 was followed for the competitive binding assay and compounds PK11195, PBR28, GE180 and the enantiomers of Compound 1 were used at 11 serial dilutions ranging from 100 μM to 1 nM. All the compounds were tested in triplicate in the [³H]PK11195 competitive binding assay and the affinity of the compounds was determined by analyzing the data using GraphPad Prism 5.0 and the low:high affinity ratios were calculated.

| | Low Affinity Site | High Affinity Site | |
|---|---|---|---|
| Compound | (nM) | (nM) | Low:High |
| PK11195 | 6 | 4 | 1 |
| PBR28 | 117 | 4 | 28 |
| GE180 | 23 | 7 | 3 |
| Compound 1 E2 | 4 | 3 | 1 |
| Compound 1 E1 | 31 | 15 | 2 |

*E1 = R enantiomer;
E2 = S enantiomer

1. A compound of the following structure: 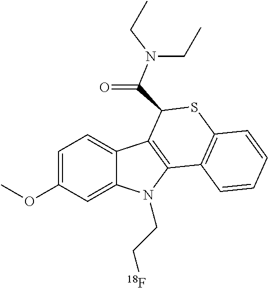 [SMILES: CCN(CC)C(=O)[C@H]1Sc2ccccc2-c2c1c1ccc(OC)cc1n2CC[18F]]
or a salt or solvate thereof.

2. A precursor compound for use in the preparation of the compound as defined in claim 1 wherein said precursor compound is of Formula I: 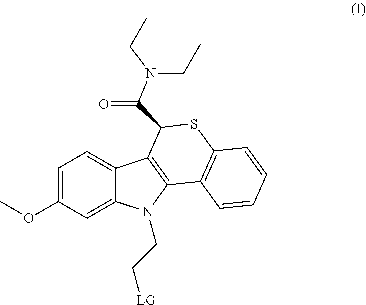
or a salt or solvate thereof;
wherein LG is a leaving group.

3. The precursor compound as defined in claim 2 wherein LG is chloro, bromo, iodo, tosylate (OTs), nosylate (ONs), mesylate (OMs) or triflate (OTf).

4. A method to prepare the compound as defined in claim 1 comprising reacting the precursor compound of Formula I with a suitable source of [¹⁸F]fluoride to obtain said compound.

5. The method as defined in claim 4 which is automated.

6. A cassette for carrying out the method as defined in claim 5 comprising:
(i) a vessel containing the precursor compound; and,
(ii) means for eluting the vessel of step (i) with a suitable source of [¹⁸F]fluoride.

7. The cassette as defined in claim 6 which additionally comprises:
(iii) an ion-exchange cartridge for removal of excess [¹⁸F]fluoride; and/or
(iv) one or more solid phase extraction cartridges for purification of the [¹⁸F] labelled reaction mixture.

8. A radiopharmaceutical composition comprising the compound as defined in claim 1 together with a biocompatible carrier in a form suitable for mammalian administration.

9. An in vivo imaging method for determining the distribution and/or the extent of translocator protein (TSPO) expression in a subject comprising:
(i) administering to said subject a compound as defined in claim 1;
(ii) allowing said compound to bind to TSPO expressed in said subject;
(iii) detecting signals emitted by the radioisotope of said compound using positron-emission tomography (PET);
(iv) generating an image representative of the location and/or amount of said signals; and,
(v) determining the distribution and extent of TSPO expression in said subject wherein said expression is directly correlated with said signals emitted by said compound.

10. The in vivo imaging method as defined in claim 9 which is carried out repeatedly during the course of a treatment regimen for said subject, said regimen comprising administration of a drug to combat a TSPO condition.

11. The compound as defined in claim 1 for use in an in vivo imaging method.

12. The compound as defined in claim 1 for use in the manufacture of a radiopharmaceutical composition for use in an in vivo imaging method.

13. A method for diagnosis of a condition in which TSPO is upregulated comprising the in vivo imaging method as defined in claim 9, together with a further step (vi) of attributing the distribution and extent of TSPO expression to a particular clinical picture.

14. The compound as defined in claim 1 for use in a method for diagnosis.

15. The compound as defined in claim 1 for use in the manufacture of a radiopharmaceutical composition for use in the method for diagnosis.
